(19) United States
(12) Patent Application Publication
(10) Pub. No.: US 2026/0232258 A1
(43) Pub. Date: Aug. 13, 2026

(54) SYSTEM AND METHOD OF CHARACTERISATION OF MAGNETIC RESONANCE MEDICAL SCANS OF THE PELVIC REGION

(71) Applicant: PERSPECTUM LIMITED, Oxfordshire (GB)

(72) Inventors: Amy HERLIHY, Oxfordshire (GB); Michael John BRADY, Oxfordshire (GB)

(21) Appl. No.: 19/470,739

(22) PCT Filed: Mar. 27, 2024
(86) PCT No.: PCT/IB2024/052937

§ 371 Date: Sep. 29, 2025

(30) Foreign Application Priority Data
Mar. 28, 2023 (GB) 2304513.1
Sep. 25, 2023 (GB) 2314650.9

(51) Int. Cl. A61B 5/00; A61B 5/055

(52) U.S. Cl. CPC A61B 5/4325; A61B 5/055

(57) ABSTRACT

A method and apparatus for analysing one or more MR images showing a pelvic region to identify features indicating areas that contain endometrial tissue is described. The method comprising the steps of: acquiring one or more quantitative non-contrast MR medical image scans of the pelvic region; determining one or more MR metrics to determine one or more multiparametric MR map for the pelvic region; and analysing the multiparametric MR map to determine a biomarker indicating regions of abnormal iron concentration.

### FIELD OF THE INVENTION

This invention relates to the use of medical scan images, particularly MR (magnetic resonance) images for the analysis of organs in the pelvic region.

### BACKGROUND

Endometriosis is a pathological condition where endometrial tissue is found outside the uterine cavity. After each menstrual cycle, the out-of-place tissue bleeds and becomes inflamed. Over time, this repeated inflammation can lead to the formation of scar tissue. Being an oestrogen dependent condition, endometriosis mainly affects women of childbearing age with a symptomatic prevalence of 10%. An additional 50% of women suffering from chronic pelvic pain will also eventually be diagnosed with endometriosis. It is estimated that 1.5 million women in the UK are affected by endometriosis.

Additionally, endometriomas (cystic lesions due to endometriosis imbedding on the ovary) can form. In this case, the cyclical monthly bleed is trapped within the ovarian structure and blood slowly builds up and over time the contents are degraded. This process leads to the formation of “chocolate cysts” which are cysts that contain a dark and gelatinous material surrounded by a fibrotic wall. Such cysts, if left untreated, could lead to infertility or ovarian cancer.

Endometriosis is commonly associated with significant pelvic and abdominal pain during menstruation (dysmenorrhoea); painful intercourse (dyspareunia); and spontaneous pain outside menstrual periods. The clinical impact of endometriosis on a woman's health can be considerable especially in the context of infertility. A substantial minority, approaching one half, of women experiencing infertility will be diagnosed with Endometriosis (Endometriosis Society).

Women with endometriosis typically present to general practitioners (GPs) with pain, and then are referred to secondary care for diagnosis and management. A significant proportion of women present to fertility services where a diagnosis is made. As per National Institute of Clinical Excellence (NICE) guidelines, diagnosis is usually made by laparoscopic inspection of the pelvis, but other less invasive methods may be used, including ultrasound and MRI scanning. Once the diagnosis is suspected, management options for endometriosis include pharmacological, surgical, and non-medical treatments.

While minor and moderate endometriosis can be managed in secondary level gynaecology departments, more severe cases necessitate complex surgery, for example in the United Kingdom, in specialist Endometriosis Centres.

In the United Kingdom, for example, endometriosis is one of the most common gynaecological conditions needing treatment. Whilst its true prevalence is often underestimated, it is believed to affect at least 1 in 10 women in the UK and a total of 190 million women worldwide. Within the NHS (National Health Service) endometriosis costs the UK economy £8.2bn a year in treatment, loss of work and related healthcare costs.

Numerous studies have been devised to study the specific costs of endometriosis to the healthcare system. A recent prospective, multi-centre international collaboration, questionnaire-based survey measured costs and quality of life in ambulatory care and in twelve tertiary care centres in ten countries. The study enrolled women with a diagnosis of endometriosis and at least one centre-specific contact related to endometriosis-associated symptoms. The main outcome measures were direct healthcare costs, cost of productivity loss, total costs, and quality-adjusted life years. The average annual total cost of endometriosis per woman was £8900. Healthcare costs were mainly due to surgery (29%), monitoring tests (19%), hospitalization (18%) and physician visits (16%). Of note, decreased quality of life, often due to delay in diagnosis, was the most important predictor of direct healthcare and total costs. Costs increased in line with the severity of endometriosis, presence of pelvic pain, presence of infertility and a higher number of years since diagnosis.

With improvement in surgical techniques and referral pathways, the prevalence of a subclass of endometriosis, known as deep infiltrating endometriosis (DIE), has been increasingly documented. Accounting for 1% to 2% of the overall prevalence of endometriosis, it is defined as endometriosis infiltrating the peritoneum by >5 mm. It is characterized by endometriotic nodules infiltrating pelvic tissues such as the uterosacral ligaments (USL), uterovesical fold, rectovaginal septum (RVS) and/or bladder. FIG. 10 shows common locations of endometriosis within the pelvis and the abdomen. Several imaging techniques, including ultrasound, computerised tomography (CT) and magnetic resonance imaging (MRI), have been previously suggested for the detection of DIE.

MRI can be used to identify endometriosis lesions. Generally, a radiologist reads, that is interprets, the scans, though this is a difficult task even for experienced, specialist radiologists. FIG. 11 shows an MRI scan where the anatomical structures within the female pelvis are identified. For example, it is often difficult to identify superficial endometriosis lesions as they are very small.

Frequently, reading times can be long and it is possible, even often the case in practice, that some lesions are missed. Currently MRI is used as a second scan after transvaginal ultrasound, but many women find transvaginal ultrasound painful not least because of the endometriosis lesions and adhesions. Other women are reluctant to have transvaginal examinations. For all of these reasons, most women would prefer a scan, such as MRI, that is not invasive.

However, this known prior art in imaging for endometriosis has the following limitations and disadvantages, particularly for DIE. The two imaging modalities most frequently used to diagnose endometriosis are transvaginal ultrasound (TVUS) and MRI (which has largely superseded CT, particularly for the diagnosis of DIE). Though ultrasound has high sensitivity and specificity for ovarian endometriosis, it is much less reliable for DIE, which often has fibrotic nodules deep within the pelvis. These significantly challenge diagnostic accuracy, depending both on the location of the lesions and the experience of the sonographer. Despite its advantages (as noted above), currently there is no single standardized MRI scanning protocol that provides all the information required for definitive clinical diagnosis. As a result, radiologists typically compare the images provided by a range of MRI pulse sequences, each taken in a variety of directions, and rely heavily on their experience. As a particularly challenging example, bladder endometriosis lesions, often present as bladder wall thickening which is frequently missed, even by an experienced operator.

Delayed diagnosis is a significant problem for women with endometriosis. Patient self-help groups emphasise how frequently healthcare professionals delay making a diagnosis, often because those professionals do not consider endometriosis as a possibility. Definitive diagnosis of endometriosis currently relies on laparoscopic pelvic inspection with histological confirmation. The current high threshold for laparoscopic examination of healthy women allied with failure of inexperienced operators to recognise the tell-tale signs of deep pelvic nodules means the time from clinical suspicion to diagnosis can be significantly delayed. The latest report from the NICE states that, in the United Kingdom, there is typically a time delay of around 7.5 years before a confirmed diagnosis of endometriosis is made. Globally, the delay can span a decade (Endometriosis Society). Furthermore, preoperative diagnosis of intestinal lesions in women with endometriosis is also critical to ensure optimal surgical debulking of deep endometriotic lesions and reduce the risk of intraoperative diagnosis which can lead to increased risks of intestinal resection, stoma, and complications.

Previously, studies assessing the predictive performance of TVUS and MRI have been shown to have a sensitivity and specificity of 90%. However, it is worth noting that the diagnostic performance of these examinations is almost certainly overestimated because of the high level of expertise of the radiologists involved in the studies compared to those in everyday practice. These studies were conducted within major tertiary units where the level of expertise surpasses that at most gynaecology centres in the United Kingdom (or worldwide).

Evidently, there is a substantial need to improve the detection of DIE lesions, and in particular to improve detection in MR images.

Thus, the following problem(s) has (have) been resolved:
Provide a non-invasive quantitative imaging method to identify endometriomas and other lesions arising from endometriosis, particularly deep infiltrating endometriosis (DIE).
Provide a non-invasive quantitative imaging method to identify pelvic lesions.
Automate identification of lesions to aid the radiologist as a decision support tool to identify endometriomas, lesions arising from endometriosis and other pelvic lesions.

### SUMMARY OF THE INVENTION

In a first aspect there is provided a method of analysing one or more MR images showing a pelvic region to identify features indicating that contain endometrial tissue comprising the steps of: acquiring one or more quantitative non-contrast MR medical image scans of the pelvic region; determining one or more MR metrics to determine one or more multiparametric MR map for the pelvic region; and analysing the multiparametric MR map to determine a biomarker indicating regions of abnormal iron concentration.

In an optional example the one or more MR metrics comprise one or more of T1, corrected T1, T2, T2*, proton density fat fraction (PDFF) and apparent diffusion coefficient (ADC).

Further preferably, the biomarker for identifying endometriomas (endometriotic cysts) is determined as one or more of: a T2* value greater than 15 ms; a T1 value less than 1000 ms. Further preferably, the one or more MR metrics further comprises a deoxyhaemoglobin/oxyhaemoglobin ratio, BOLD/R2*.

In an optional example the determined metrics generate an MRI map for each of the determined metrics.

In a further example, at least two of the determined metrics are combined to generate a further metric map, and this further metric map is used in the calculation of the pelvic composite map.

In an optional example raw values for the at least two determined metrics are used to generate the further metric map.

In a further preferred example the regions of characteristic T2* values indicate the presence of endometrial tissue in the pelvic region.

In an optional example the method comprises determining the age of the endometrial tissue using the T2* value.

In an optional example, the one or more MR medical scan images are obtained at a clinical magnetic field strength. Preferably, the field strength is at least 1.5T, further preferably, the field strength is 3T.

In a further example, the MRI scan(s) is/are a 2D scan(s) which produces a set of 2D images comprised of pixels, or 3D scan(s) with a 3D image comprised of voxels.

In a further example, the MRI metric map is a PDFF map, T1 map, or a corrected T1 map.

In an optional example, at least one MRI metric is combined with one or more other MRI metrics, before generation of an additional MRI metric map which provides an improved estimate of the initial value of at least one MRI metric.

In an optional example, the at least one metric is a T1 metric, and is combined with at least one of PDFF and T2*.

In a further example, the method further comprises the step of generating at least one of the following MRI metric maps: T1 map, T2 map, T2* map, ADC map.

In an optional example, the T1 metric is determined using a Modified Look Locker Inversion recovery sequence acquisition, shortened Modified Look Locker Inversion recovery sequence acquisition, a variable flip angle sequence acquisition.

In a further example the T2* metric and/or the PDFF metric are determined using a multi echo gradient echo sequence acquisition.

Further preferably, the T2 metric is determined using a multi-contrast spin echo sequence acquisition.

In a further example, the ADC metric is determined using a single shot or multi-shot diffusion weighted Echo Planar Imaging.

In an optional example, MRI images are acquired over a set time period to monitor changes in the pelvic region. Preferably, the set time period is a minimum of 1 week. In an optional example, the MRI scan is a scan of the entire pelvic region.

In a second aspect there is provided an apparatus for analysing MRI data from a scan of the pelvic region to determine regions of endometriotic lesions, the apparatus comprising at least one processing component arranged to perform the method of any preceding claim.

### BRIEF DESCRIPTION OF THE FIGURES

The invention will now be described, by way of example only, with reference to the accompanying figures in which:

FIG. 1 is a flow chart of the steps of the method according to an example;

FIG. 2 shows the flow chart to generate a T1 map using one of many appropriate methods;

FIG. 3 shows a flow chart for a T2* map calculated using a multi-echo gradient method;

FIG. 4 shows a flow chart for a T2 map calculated using multi contrast spin echo pulse sequence;

FIG. 5 shows a flow chart for diffusion weighted EPI (echo planar imaging);

FIG. 6 shows the calculation of proton density fat fraction (PDFF);

FIG. 7 shows T2* MRI image according to an example;

FIG. 8 shows T1 MRI image according to an example;

FIG. 9 shows PDFF MRI image according to an example;

FIG. 10 shows a drawing of the female pelvis, indicating common locations of endometriosis;

FIG. 11 shows an MRI image where anatomical structures in the female pelvis are labelled;

FIGS. 12(a)-(c) show T2 weighted images for the atlas according to an example;

FIGS. 13(a)-(c) show T1 weighted (VIBE) images for the atlas according to an example;

FIG. 14(a) shows a T1 vs T2*plot of a range of tissues found in the female pelvis;

FIG. 14(b) shows a T1 vs PDFF plot of a range of tissues found in the female pelvis;

FIG. 14(c) shows a T2* vs PDFF plot of a range of tissues found in the female pelvis;

FIG. 15 shows manual selected regions of interest of various tissues in the female pelvis, the anatomical scan of the same slice and demonstration of a map indicating various tissues, grouped by their T1, T2* and PDFF values; and

FIG. 16 shows the T1 map, T2* map and PDFF map of the same slice used in FIG. 15 to demonstrate identifying tissue with MRI metric values (T1, T2* and PDFF).

### DETAILED DESCRIPTION OF THE INVENTION

The present invention will now be described with reference to the accompanying drawings in which there is illustrated an example of a method and apparatus for characterization of organs and tissue in the pelvic region, from medical imaging scan data. The method uses quantitative MR imaging, preferably, without requiring use of contrast agents. However, it will be appreciated that the present invention is not limited to the specific examples herein described and as illustrated in the accompanying figures.

In the figures like reference numbers are used to identify like or functionally similar elements. Elements in the figures are illustrated for simplicity and clarity and have not necessarily been drawn to scale.

Furthermore, because the illustrated embodiments of the present invention may, for the most part, be implemented using components known to those skilled in the art, details will not be explained in any greater detail than that considered necessary as illustrated below, for the understanding and appreciation of the underlying concepts of the present invention and in order not to obfuscate or distract from the teachings of the present invention.

By using quantitative MRI methods, it is possible to measure different tissue metrics, these include but are not limited to T1, T2, T2*, PDFF, and ADC. Quantitative MRI methods have been used over the years to investigate tissue and disease states. In the early 1980s investigation of tissue characteristics such as T1 and T2 were important to help develop MRI methods that provided images with tissue contrast that highlights pathology. The original “gold standard” quantitative MR methods such as Inversion Recovery (IR), are intrinsically too slow for clinical use: not only will patients not tolerate lying in the scanner for extended periods; but those sequences are prone to the inevitable patient motion during the extended scan period.

Improvements in both MRI hardware, computer systems and pulse sequences mean that there are now a variety of methods to quantitively measure tissue characteristics. Such newer methods enable quantitative images to be collected in a single breath-hold (a breath-hold scan). It should also be noted that multiple methods have been developed for measuring each of the MR tissue metrics. For example, MOLLI, (Modified Look-Locker Inversion Recovery), sh-MOLLI (shortened MOLLI) as described in U.S. Pat. No. 10,228,432 and GB 2497668 and Variable Flip Angle (VFA) methods all enable accurate estimation of the T1 parameter. Note that all such methods provide estimates of the corresponding physical parameter. For example, T1 values estimated using MOLLI are generally consistently lower than the “gold standard” (clinically infeasible IR), while T1 values estimated using VFA techniques are consistently higher.

As with T1, there are a variety of methods to measure other quantitative metrics. For example, the estimation of T2 typically requires the use of one of several pulse sequences that provide spin echo data. Similarly, the estimation of T2* typically requires the use of one of several multi-echo gradient echo pulse sequences. Again, the estimation of PDFF (proton density fat fraction) is typically collected with a multi-echo gradient echo pulse sequence, though the pulse sequence settings can differ between the estimations of T2* and PDFF. As is readily appreciated by those skilled in the art, similar considerations apply to Apparent Diffusion Coefficient (ADC) maps, which measures the magnitude of the diffusion of water molecules in a tissue. Changes in ADC are correlated, for example, with clinical deficits in tissue. The estimation of ADC typically involves single shot or a multi shot diffusion weighted EPI (echo planar imaging), though other methods are known.

In all cases, the data needed to generate the quantitative maps of relevance for endometriosis necessitate a plurality of pulse sequences appropriate to each parameter of interest.

It is also known that faster quantitative MRI scanning methods may be impacted by confounding information from other tissue components. For example, it is known that measuring T1 using MOLLI in an environment that has very short T2* (such as a liver with high iron) results in T1 values that are lower than if measured by a (clinically infeasible) ground truth method. Techniques have been developed to correct for such bias in MOLLI T1 values for short T2*: see for example, patent number GB2497668. Likewise, tissue with high fat content impacts T1 measurements using MOLLI. Therefore, it is appreciated that care must be taken in collecting quantitative MRI metrics, ensuring not only that clinically useful methods are deployed; but also that appropriate correction methods are applied. Using existing tools for fast quantitative MR imaging and correction for confounders, it is possible to utilise quantitative imaging to characterise tissue in the clinic.

In normal practice each quantitative MR measurement is made on a pixel-by-pixel (2D scan) or voxel-by-voxel (3D scan) basis. If the data is regularised, then the metric can be improved to reduce factors such as noise. Standard image analysis tools can be used to ensure that quantitative parametric maps provide optimal estimates of the desired metrics.

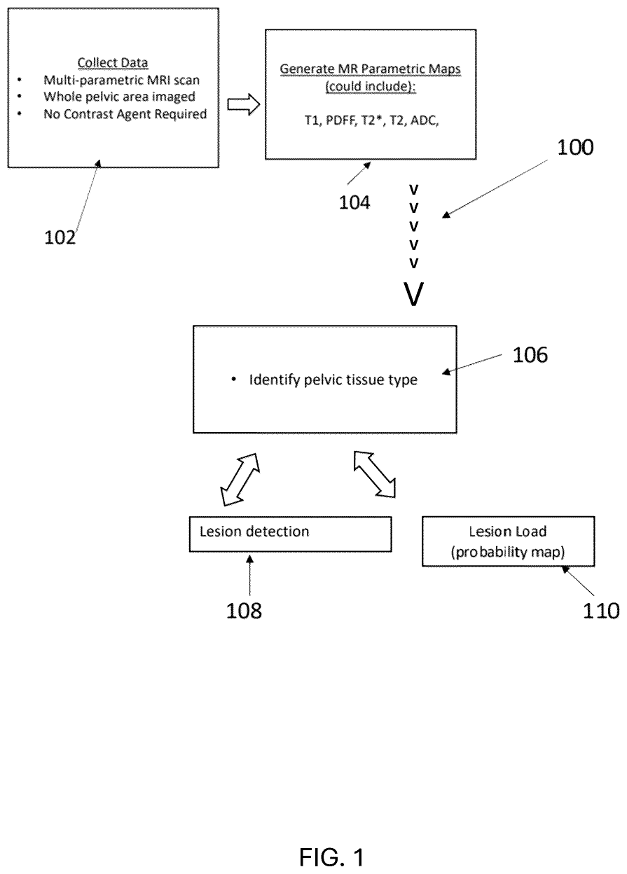

FIG. 1 shows a flow chart of the steps of the method 100. Preferably, a method of analysing one or more MR images showing a pelvic region to identify features indicating a region of abnormal iron concentration, which may indicate endometriosis lesions concentration comprising the steps of: acquiring one or more quantitative non-contrast MR medical image scans of the pelvic region; determining one or more MR metrics to determine one or more multiparametric MR map for the pelvic region; and analysing the multiparametric MR map to determine a biomarker indicating regions of biomarkers of abnormal tissue, including abnormal iron concentration (where the iron concentration is either higher than expected, or lower than expected). Preferably, the regions of abnormal iron concentration indicate a presence of endometrial tissue in the pelvic region. At step 102, an MRI scan is completed to provide multi-parametric data, preferably with coverage of the entire pelvic region, although the coverage may be of a selected part of the pelvic region. The MRI scan is a quantitative scan, and in a preferred example, the scan is performed without contrast agents. Contrast enhanced MRI scans utilise an exogenous, typically injectable compound, that changes the MRI characteristics of a tissue, including blood. Although this can provide information about where the contrast agent can be found within tissues, or rates of change in signal intensity due to the contrast, contrast enhanced MRI does not provide information about the native MRI characteristics of tissues, therefore the preferred example is a non-contrast enhanced scan. In an example a plurality of quantitative MRI scan images of at least part of the pelvic region area are acquired using different MR pulse sequences for each MRI scan image The acquired MRI scans are either 2D scans which produces a set of 2D images comprised of pixels, or 3D scans with a 3D image comprised of voxels. A 2D scan will produce 2D maps, and a 3D scan will produce 3D maps. At step 104 one or more MR parametric maps are generated from some or all of the data in step 102.

Step 104 then generates one or more MR parametric maps from the one or more MRI metrics from MR scan data by using appropriate fitting algorithms. Preferably, the determined metrics are used to generate an MR parametric map for each of the determined metrics. The MR parametric maps may include one or more of T1, corrected T1, T2*, T2, PDFF and ADC. Step 106 is analysis of at least one of the MR metric maps to generate one or more of pelvic region MR maps, to show tissue characteristics of fat and non-fat tissue in the MRI image, and to identify the tissue type in the image. Preferably, the one or more MR metrics comprise one or more of T1, corrected T1, T2, T2*, PDFF and ADC. In an example at least two of the determined metrics are combined to generate a further metric map, and this further metric map is used in a calculation of a pelvic composite map. Further preferably, raw values for the at least two determined metrics are used to generate the further metric map.

Step 108 is the detection of possible endometria lesions in the image, and step 110 allows determination of lesion load as a probability map. As explained in more detail below the analysis is used to determine regions with specific MRI biomarker values that can be used to indicate the presence of an endometrioma, indicated by the presence of an abnormal iron concentration. In an example MR images are acquired over a set period of time to monitor changes in the pelvic region. Preferably, the set time period is a minimum of 1 week.

Quantitative MRI is used to generate multiparametric maps in order to identify area(s) in the pelvic region that have an abnormal iron concentration, that may indicate the presence of endometriosis lesions, for example areas that may be endometriomas. A variety of these metric maps, including, but not be limited to: T1 and T2* are used. As noted above, additional MRI metrics may include T2, PDFF, diffusion or other quantitative MRI metrics. In a preferred example, at least one MRI metric is combined with one or more other MRI metrics, before generation of an additional MRI metric map which provides an improved estimate of an initial value of at least one MRI metric.

The at least one metric may be a T1 metric, and may be combined with at least one of PDFF and T2*.

The MRI measurements will be collected using an MRI scanner (typically 1.5T or 3T, though the magnetic field strength could be any clinical magnetic field strength). The scan will utilise standard MRI radiofrequency coils.

In a preferred example, the MRI scan volumes (either multi-slice 2D or 3D scans to produce 2D and 3D maps, respectively) to preferably fully cover the pelvic region are acquired at step 102. The MRI scan volumes are collected for quantification of one or more of the following MRI parameters or metrics.
metric cT1 is a T1 measurement that is standardized for iron content in the liver and standardized across vendors and field strengths, and is described in GB2497668. The corrected T1, (i.e., cT1) will be standardized across vendors and field strengths and corrected for fat content. In an example a standardization method is set up by establishing a “ground truth” scanner system and then performing the quantitative scans across the scanner manufacturers (typically GE, Philips, and Siemens) to generate a standardization table to map phantom data back to the values measured on the “ground truth” scanner.
Metric T1 using an MR pulse sequences such as Modified Look-Locker Inversion Recovery (MOLLI), sh-MOLLI and/or Variable Flip Angle method. In a preferred example the T1 metric is determined using a Modified Look Locker Inversion recovery sequence acquisition, shortened Modified Look Locker Inversion recovery sequence acquisition a variable flip angle sequence acquisition, or other quantitative method.

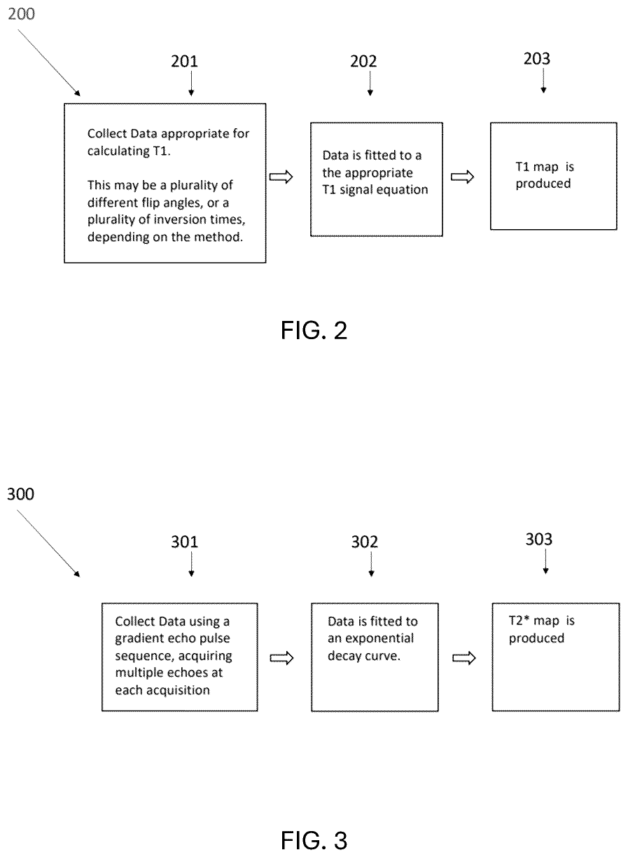

Determination of a T1 map from a MOLLI pulse sequence (also Sh-MOLLI) is shown in FIG. 2.

MOLLI (Modified Look-Locker Inversion recovery) is an inversion recovery pulse sequence 200 that collects data along the T1 recovery curve over 2 or 3 acquisition blocks. Initially, MR data is collected at step 201, this pulse sequence samples the T1 curve by collecting at least three sets of data over the T1 curve. Sh-MOLLI samples the T1 curve similarly, but with significantly shorter time scales and a reduced number of data points, enabling a shorter breath-hold MRI scan.

Example scan times are 18 sec for a MOLLI scan and 9 seconds for a sh-MOLLI scan. The collected data is then fitted to an inversion recovery T1 recovery curve at step 202. Finally the T1 metric map is produced at 203. Sh-MOLLI (shortened) Modified Look-Locker Inversion recovery, follows the same general steps, just over a shorted time frame. Both of these sequences can be used for breath-hold scans, which reduces motion artefacts. They are very often used for cardiac T1 parametric scans.

A corrected T1 parameter can also be calculated from the raw T1 metric as shown in FIG. 2.

FIG. 3 shows the determination of a T2* metric 300 using an MR pulse sequence such as a multi-echo gradient echo pulse sequence.

Multi-echo gradient echo sequences are gradient echo sequences that have been set up to acquire different echo times during a single MRI scan-rather than running multiple gradient echo scans, each with a different echo time. Using a multi-echo gradient echo provides a time efficient method for collecting scans with different echo times needed for generating parametric maps. Gradient echo sequences may be designed in such a way that the information they collect provides data to calculate T2* maps, which takes into account the magnetic field around the tissue structure. Initially, data is collected using a gradient echo pulse sequence at step 301, acquiring multiple echoes at each acquisition, then the acquired data is fitted to an exponential decay curve at step 302. Finally, a T2* map is produced at step 303. In an example the PDFF parameter and metric map are determined using multi-echo gradient echo pulse sequence. In a preferred example, the T2* metric and/or the PDFF metric are determined using a multi echo gradient echo sequence acquisition.

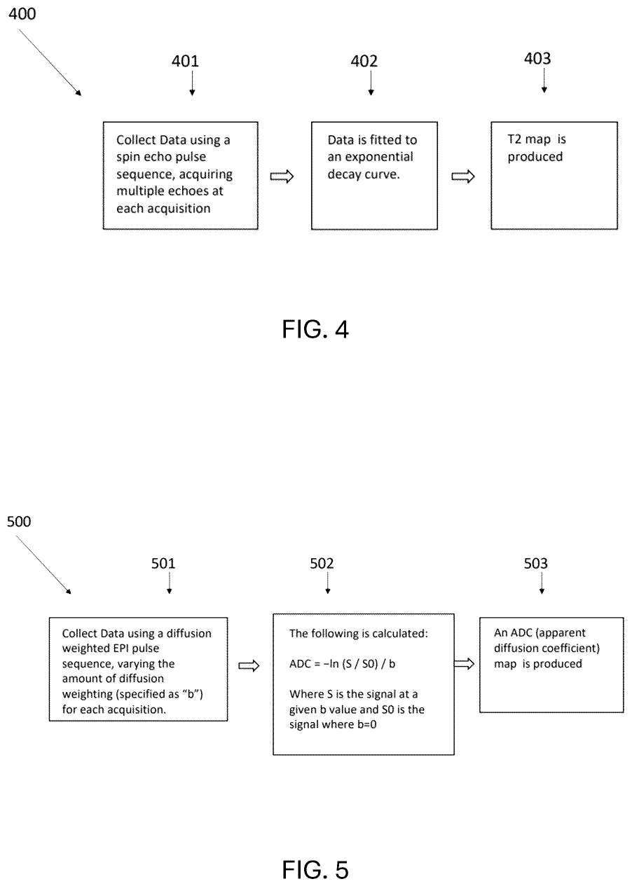

FIG. 4 shows the determination of a T2 metric 400 using an MR pulse sequence such as a multi-contrast spin echo pulse sequence.

Multi-contrast spin echo sequences are spin echo sequences that are set up to acquire different echo times during a single MRI scan. As with the multi-echo gradient echo, this provides a time efficient method for collecting spin echo data with different echo times. Spin echo sequences are designed in such a way that the information they collect provides data to calculate T2 maps. The magnetic field does not have an impact on these scans. Where T2 is a characteristic of the tissue, T2* is a characteristic of the tissue within its physical environment. At step 401 data is collected using a spin echo pulse sequence, acquiring multiple echoes at each acquisition. Step 402 follows where the acquired data is fitted to an exponential decay curve. Finally, at step 403, a T2 map is produced. In an example the T2 metric is determined using a multi-contrast spin echo sequence acquisition.

FIG. 5 is relevant to a diffusion weighted EPI (echo planar imaging) method. FIG. 5 illustrates the determination of an Apparent Diffusion Coefficient (ADC) metric 500, using MR pulse sequences such as a single-shot or multi-shot diffusion weighted Echo Planar Imaging (EPI). The ADC metric is a parameter which measures the magnitude of the diffusion of water molecules in a tissue.

At step 501, Data is collected using a diffusion weighted EPI pulse sequence, varying the amount of diffusion weighting (specified as “b”) for each acquisition. Step 502 follows, where the following is calculated:

$$ADC = - ln ( S / S 0 ) / b$$

where S is the signal at a given b value and S0 is the signal where b=0.

Finally, in step 503, an ADC map is produced. Preferably, the ADC parameter is determined using a single shot or a multi shot diffusion weighted echo planar imaging. EPI pulse sequences are extremely fast scans that can take less than a second to acquire. The addition of diffusion weighting to a pulse sequence is an option that is very time consuming, such that diffusion weighting on spin echo scans, can mean the scans become too long for clinical use. Therefore, diffusion weighted scans tend to be based around EPI pulse sequences. The added diffusion weighting provides information, the value ADC (Apparent Diffusion Coefficient) related to how water molecules are diffusing in the tissue.

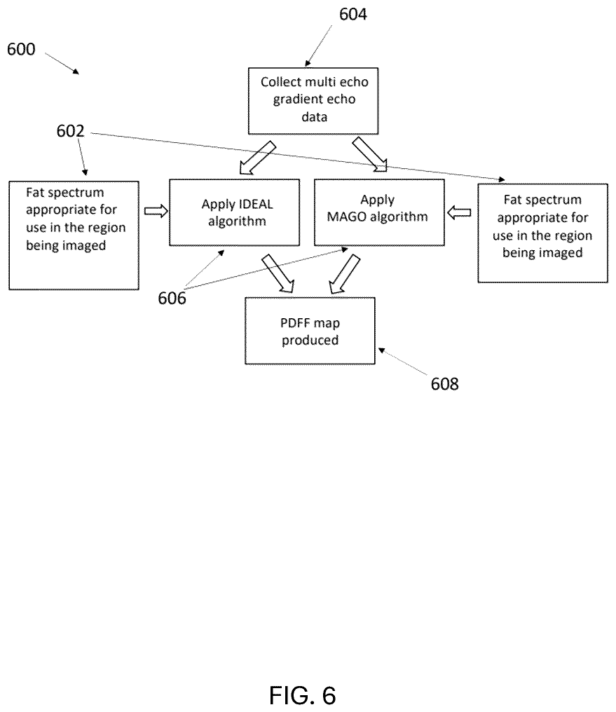

In an example, the calculation of proton density fat fraction (PDFF) 600 from the scan data for the pelvic region is performed, as illustrated in FIG. 6 . . . . A fat spectrum appropriate for use in the pelvic region is used in step 602, and in a preferred example, multi echo gradient echo MRI scans are acquired 604, although other pulse sequences may be used for other examples. The multi echo gradient echo data and the pelvic region appropriate fat spectrum are provided as inputs to an algorithm 606. A variety of different algorithms are available to do this, including the IDEAL method (as described in U.S. Pat. No. 7,176,683) and the MAGO method (MAGO method as described in GB2576886). The PDFF measurement that results 608 from the algorithm provides an estimation of the amount of “fat” in each voxel of the MRI image. The calculation of PDFF typically requires a MR spectrum of the fat contained in the tissue being imaged 602, although other methods of determining PDFF may be used in alternative examples. The “fat” in the MR spectrum may be one particular species of lipid or represented as a summary of all different lipid types. An MR spectrum which is appropriate for use in the pelvic region is used in conjunction with existing IDEAL or MAGO algorithms (referenced above) to produce the PDFF measurement in each voxel. These algorithms use two species within a voxel (such as fat and water) to determine the relative signal contributions within that voxel, preferably by means of a cost function analysis method although other methods may also be used.

The MRI metrics that are determined from the different pulse sequences described above are then used to generate a corresponding MRI metric map for each of the determined MRI metrics. In a preferred example, the MRI metric maps are used for calculating different MR metric maps to show tissue characteristics of fat and non-fat tissue in the MRI image, as well as provide information on ion concentration in the tissue.

Different MR pulse sequences to obtain MR metrics, as mentioned in the descriptions of the scans above, may be utilised for different reasons. This may be to optimize tissue contrast whilst maintaining a clinically relevant scan time. The different MR pulse sequences may be performed in any order, and there may be specific operating protocols for different MRI scanner manufacturers, or other reasons for example, but the order of the MR pulse sequences is not important, merely the fact that a plurality of different MR pulse sequences are used. In an example one or more MR pulse sequences may be used, and in further examples all of the different pulse sequences may be used to provide the MR metric from each different pulse sequence.

The MRI pulse sequences described above, as well as other methods to measure MRI tissue metrics, are well described in established textbooks such as:
Hashemi R H, Bradley W G, Lisanti C J. MRI: The Basics: Wolters Kluwer Health; 2012, (3rd Edition, Part II, sections 19-23);
Dale B M, Brown M A, Semelka R C. MRI: Basic Principles and Applications: Wiley; 2015, (5th Edition, Chapter 6);
Bernstein M A, King K F, Zhou X J. Handbook of MRI Pulse Sequences: Elsevier Science; 2004, (1st Edition, Chapters 14, 16, 17); and
Brown R W, Haacke E M, Thompson M R, Venkatesan R. Magnetic Resonance Imaging: Physical Principles and Sequence Design: Wiley; 1999, (1st Edition, Chapters 17, 18, 22 and 26).

As the field continues to progress, it is expected that new methods of measuring tissue characteristics (such as, but not limited to, T1, T2 and T2*). The concept described in this patent is not limited to a specific method of measuring the tissue characteristics. Any valid method of measuring the MRI characteristics will be appropriate.

Data is collected using the methods described above to generate assorted quantitative MR parametric maps.

The MR maps will typically be calculated using computer algorithms implemented to calculate the MR metrics (as described in the several references listed above) or are contained in the other publications, which includes: MAGO method (MAGO method as described in GB2576886) and cT1 (as described in GB2497668).

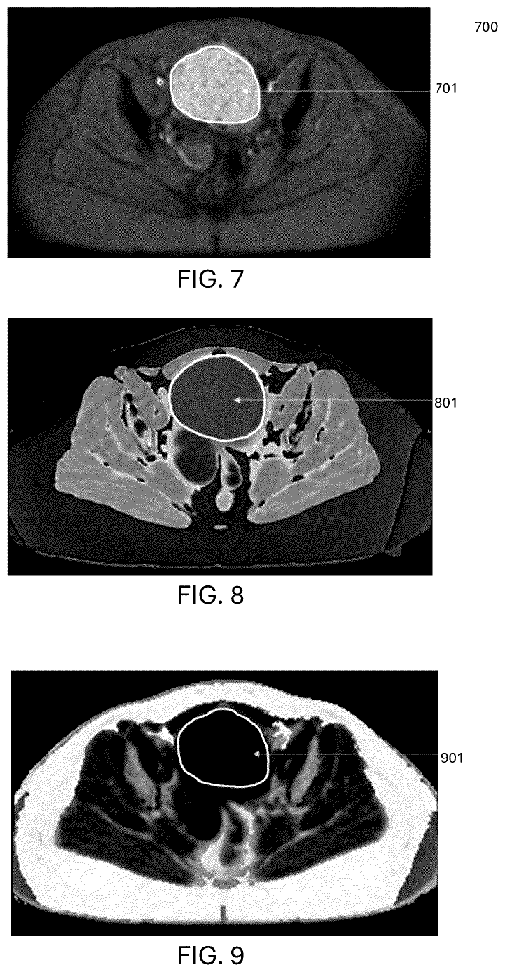

FIG. 7 shows a T2* map 700 that was obtained using the MAGO method as described in GB1814358.6 published as GB2576886. The region indicated at 701 indicates the presence of endometrial tissue in the image.

FIG. 8 is a T1 map obtained using the MOLLI method (as described in an earlier reference). The feature highlighted at 801 is the area of endometrial tissue in the image.

FIG. 9 is the PDFF map determined using the same data set as for FIG. 7. The area of endometrial tissue is shown at 901. As endometriomas have little or no fat content, this is indicated by the PDFF metric of zero or near zero in the map and shows as black. The region has been demarcated with solid white line 901.

FIGS. 7-9 show regions of endometrial tissue, shown at 701, 801, 901. These regions on the MR images have: 1) a long T2* with median of 82 ms, shown in the T2* map in FIG. 7, 2) a short T1 with median value of 500 ms, shown in the T1 map in FIGS. 8, and 2) a median PDFF value of 0%, shown in FIG. 9. It is known that T2* is a marker of iron in the liver [Ref: Wood J C, Enriquez C, Ghugre N, et al. MRI R2 and R2* mapping accurately estimates hepatic iron concentration in transfusion-dependent thalassemia and sickle cell disease patients. Blood 2005; 106:1460-1465]. In the liver, T2* value typically range from 8-30 ms, where T2*<13 ms indicate a high iron concentration. Therefore, these images with a higher T2* value indicates that there is very little iron/fresh blood in the endometrioma and the T1 value indicates that the endometriomas are not fluid filled cysts, as a cyst would typically have a T1 value of over 1000 ms, whereas this structure has a T1 of 500 ms. In an example it may be possible to determine the age of the endometrial tissue from the T2* value as, in a newly formed endometrioma, the iron content that remains from the bleed will still be high and will be indicted by a low T2* value, whereas since the blood components break down over time [Reference: Gaillard F., et al. Radiopaedia.org, https://radiopaedia.org/articles/6671] the iron content in older endometriomas will be low which will be indicated by a high T2* value.

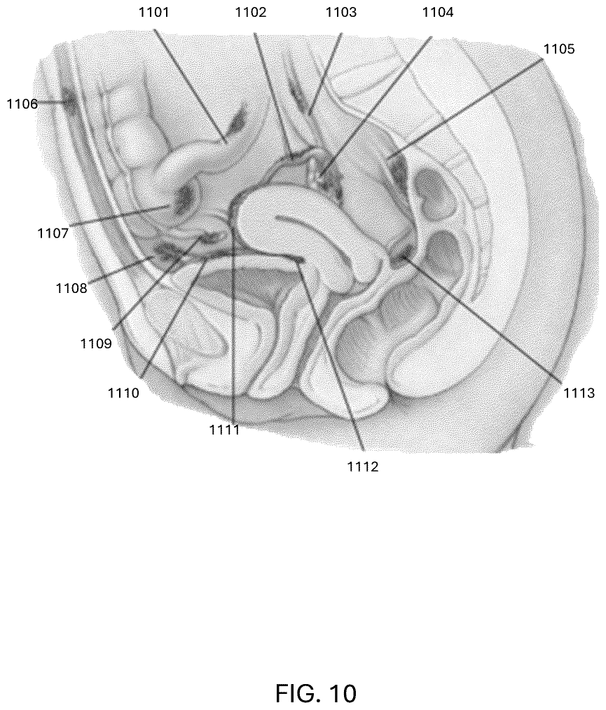

FIG. 10 shows common locations of endometriosis within the pelvis and the abdomen. Anatomical structures in the vicinity of the pelvis and abdomen include: small bowel 1101, fallopian tube 1102, ureter 1103, ovary 1104, sigmoidal colon 1105, umbilicus 1106, cecum 1107, peritoneum 1108, appendix 1109, bladder 1110, uterine serosa 1111, uterovesical fold 1112, rectovaginal septum and uterosacral ligaments 1113.

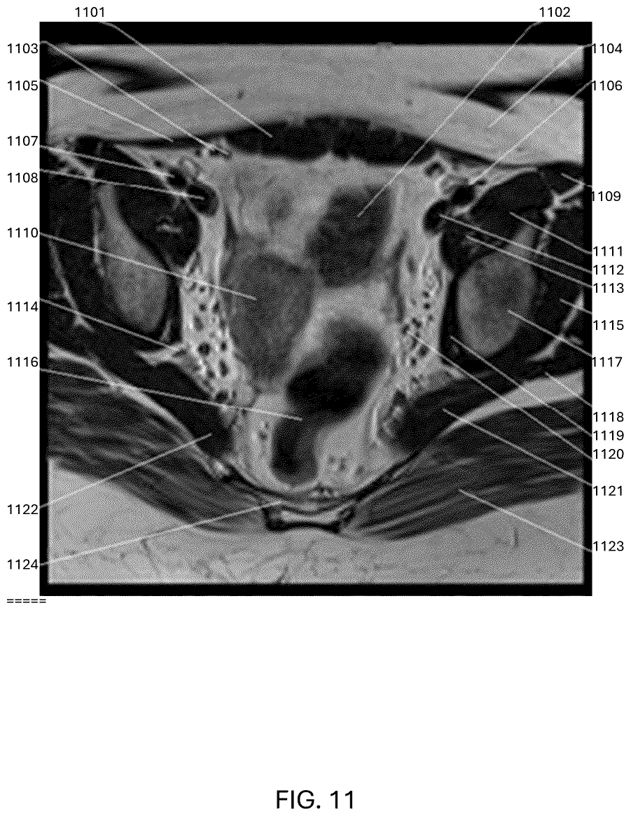

Standard anatomical MRI images are used as an atlas for aiding in identification of the anatomical regions which are contained in the parametric maps, that are obtained using the methods described above. FIG. 11 shows an MRI scan where the anatomical structures within the female pelvis are identified, including: rectus abdominus muscle 1101, sigmoid colon 1102, inferior epigastric artery & vein, adipose tissue 1104, abdominal oblique muscles 1105, left external iliac artery 1106, right external iliac artery 1107, right external iliac vein 1108, sartorius muscle, uterus 1110, iliacus muscle 1111, left external iliac vein 1112, tissue 1113, lumbosacral trunk 1114, gluteus minimus muscle 1115, sigmoid colon 1116, body of ilium 1117, gluteus medius muscle 1118, obturator internus muscle 1119, ureter 1120, piriformis muscle 1121, piriformis muscle 1122, gluteus maximus muscle 1123, sacrum 1124.

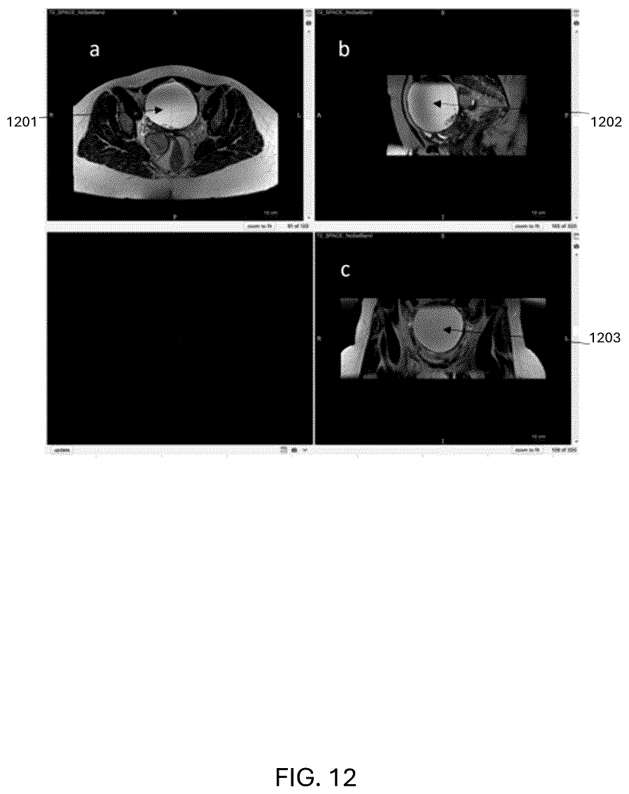

FIGS. 12(a)-(c) show T2 weighted images for the atlas. The 3 images area showing the 3 planes (axial, sagittal and coronal) of the data at the location demonstrating the endometrioma, 1201, 1202 and 1203.

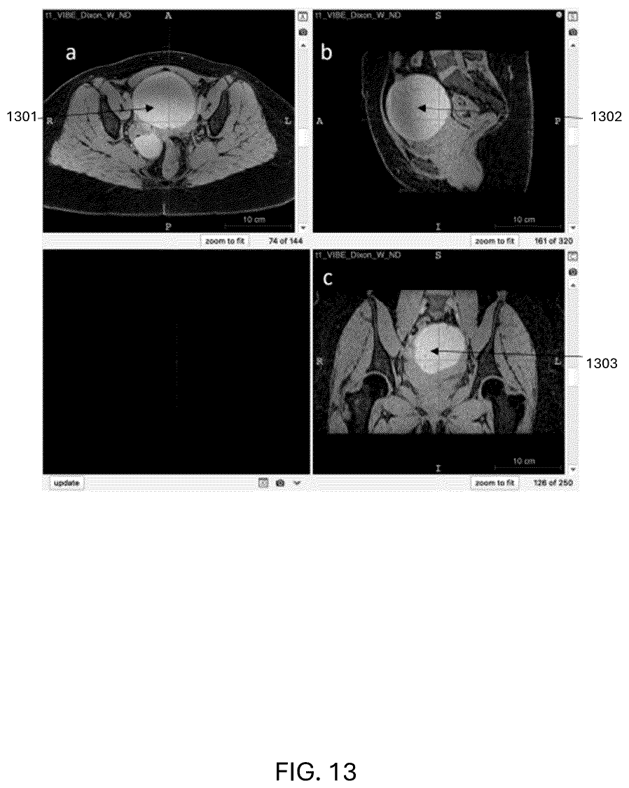

FIGS. 13(a)-(c) show T1 weighted (VIBE) 3D images 1301, 1302, 1303 for the atlas. The 3 images area showing the 3 planes (axial, sagittal and coronal) of the data at the location demonstrating the endometrioma.

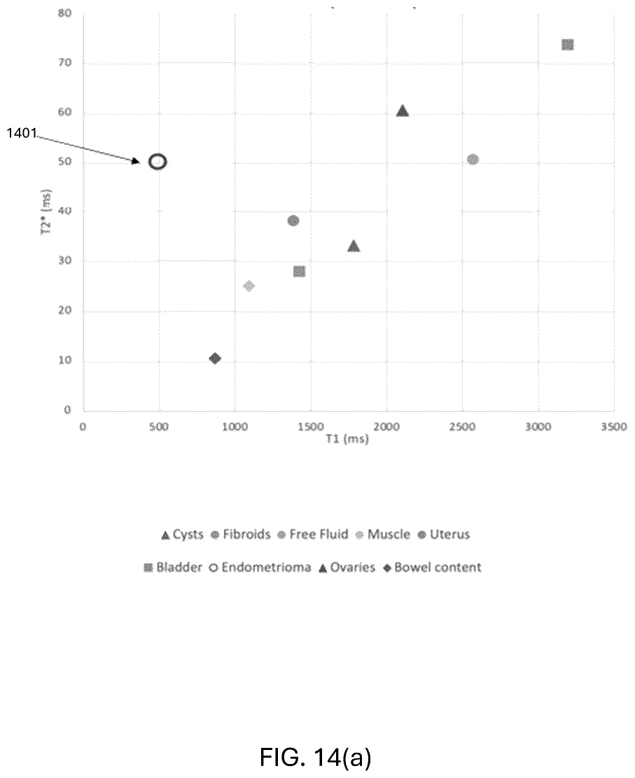

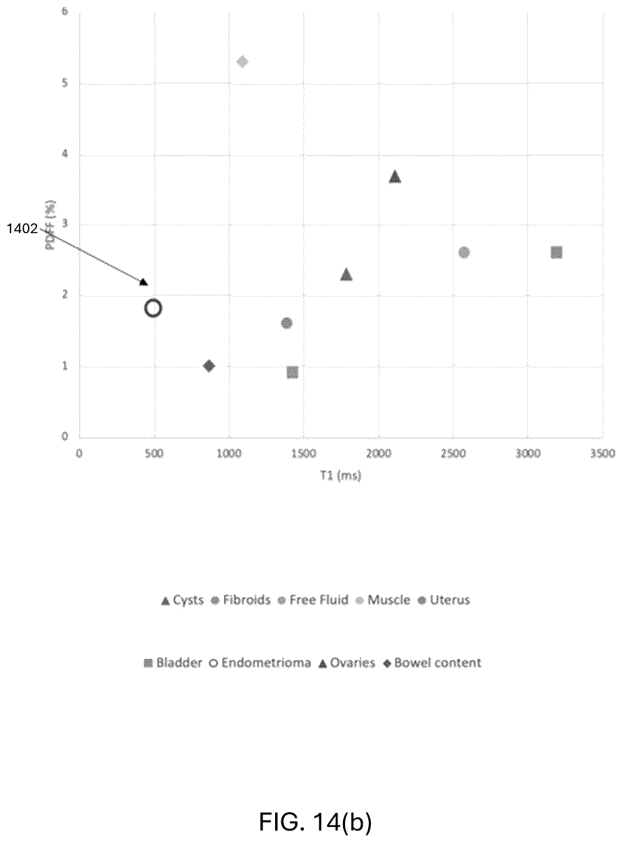

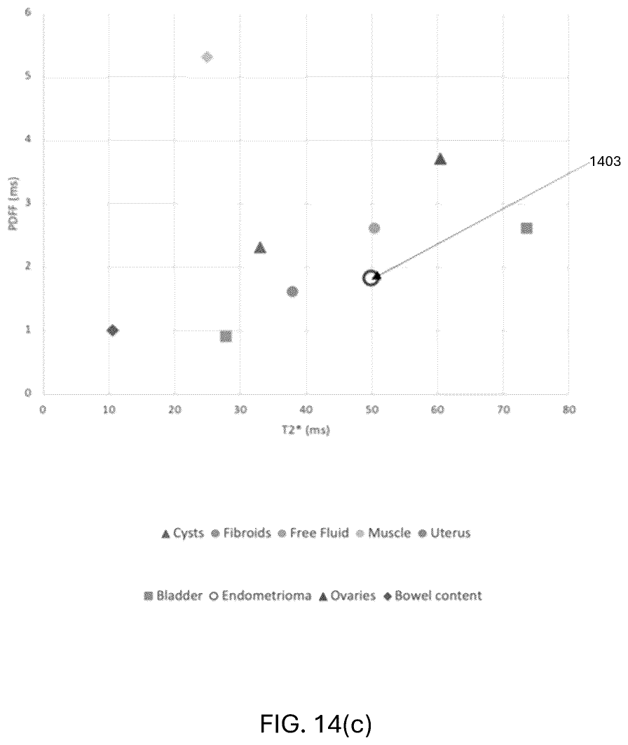

FIGS. 14(a), 14(b), and 14(c) show MRI parametric qualities of pelvic tissue. FIG. 14(a) shows a T1 vs T2* plot of tissues commonly found in the female pelvis, FIG. 14(b) is a similar plot showing T1 vs PDFF, and FIG. 14(c) shows T2* vs PDFF. These plots demonstrate that specific tissues, for example endometriomas, which are shown as the open circle (1401. 1402, 1403), have a unique quantitative MRI signal, in that the T1 and T2* values of endometriomas are not in a range similar to other pelvic tissues. This is an example of how to differentiate tissues using the biomarkers as determined from the MR images and data. As shown in these figures the Endometrioma have a T1 of 500 ms, T2* of 50 ms and PDFF of 1.8%. On the T1 vs T2* plot it can be seen that the endometrioma tissue has a unique signal district from the other tissues that were measured. Specifically, In a preferred example, for MR images obtained on a scanner at 3T a threshold of T2* to be >15 ms in combination with T1 to have a value <1000 ms, as shown in these figures enables delineation of regions of abnormal iron concentration, which may indicate the presence of endometriomas in the region that is shown on the MR images.

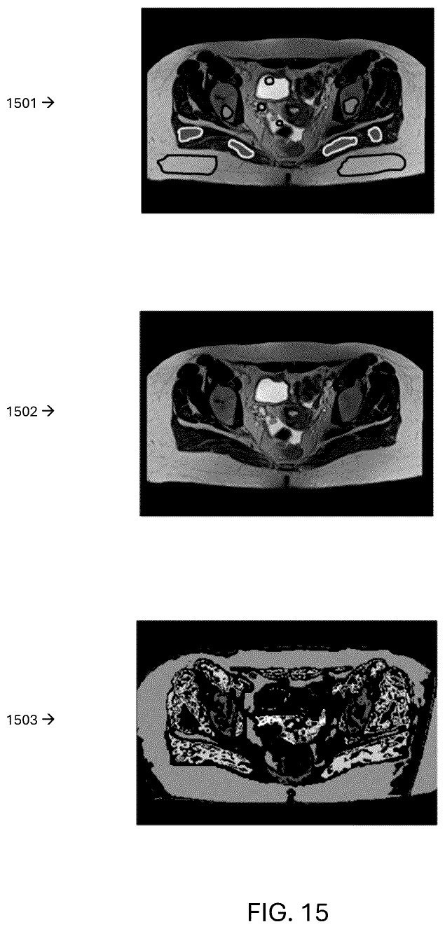

FIG. 15 shows manually selected regions of interest of various tissues in the female pelvis, the anatomical scan of the same slice and demonstration of a map indicating various tissues, grouped by their T1, T2* and PDFF values. 1501 shows example of manual regions of interest of pelvic tissues. The bold lines outline the manually selected tissue regions. 1502 is an anatomical scan, and 1503 is a demonstration of a map indicating different tissues found within the pelvis.

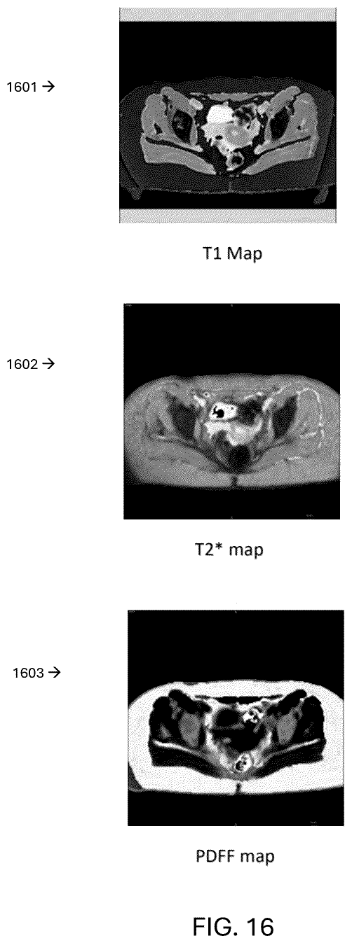

FIG. 16 shows the T1 map, T2* map and PDFF map of the same slice used in FIG. 15 to demonstrate identifying tissue with MRI metric values (1601 is the T1 map, 1602 is the T2*map and 1603 is the PDFF map). FIG. 16 shows different parametric maps of the same location within the female pelvis.

As described above, examples of the method relate to one or more novel biomarkers combining one or more quantitative values of MRI metrics such as T2*, T1 and PDFF metrics to enable detection and delineation of areas of abnormal iron concentration, which may be indicative of the presence of endometriomas. In a preferred example, for MR images obtained on a scanner at 3T a threshold of T2* to be >15 ms in combination with T1 to have a value <1000 ms enables delineation of regions of abnormal iron concentration, which may indicate the presence of endometriomas.

It is appreciated that this is only one of a family of such biomarkers, in which a specific value of T1 is paired with distinct T2* values (the latter indicating the age of an endometrioma or acute bleeds in superficial endometriosis) or in which a paired T1, T2* value to indicate superficial peritoneal endometriosis.

Additional quantitative MRI measurements beyond T2*, T1 have been incorporated in biomarkers. These include ADC, or BOLD/R2* [Imaoka I, Nakatsuka T, Araki T, Katsube T, Okada M, Kumano S, et al. T2* relaxometry mapping of the uterine zones. Acta Radiol 2012; 53:473-477.] (where BOLD/R2* is the ratio of deoxyhaemoglobin to oxyhaemoglobin in the blood). These provide metrics that can be used to indicate the location of abnormalities within the pelvic structures.

In addition, an example of the method can be integrated into the analysis of commercially available 3D MRI images (e.g., VIBE (Volumetric interpolated breath-hold examination) or LAVA (Liver Acquisition with Volume Acquisition) which are T1 weighted 3D scans collected in a breathhold or T2 weighted images). One way in which such 3D scans can be used as part of our approach to delineating endometriomas is as follows: (i) 3D computational models of “standard” anatomical structures are available: such a model is referred to as an “atlas”; (ii) using standard image analysis methods, such an atlas can be deformably registered (aligned) to a 3D image such as VIBE. This enables any of the anatomical structures to be associated with a specific region of the 3D VIBE and which this is referred to as a region of interest; (iii) the quantitative images that have been developed, even if they are 2D “slice” images, are aligned with the 3D VIBE, and so the intersection of the region of interest and the 2D slice image is known; the quantitative analysis is limited to the 2D regions defined in steps (i-iii).

As described with respect to the figures, quantitative non-contrast MRI Data required for generating one or more multiparametric maps of the pelvic region (such as T1, corrected T1, PDFF, T2, T2*, ADC and BOLD R2*) is collected. In an example the MRI metric map is a PDFF map or a corrected T1 map. Additionally, localizer scans and non-quantitative volumetric scans may also be collected for structural investigation and data overlay. The data acquired from the quantitative MRI scan is then processed as follows.

Using the quantitative MRI data collected as described above, pixel/voxel-wise MR metric maps (such as T1, corrected T1, PDFF, T2, T2* and ADC) are calculated. In an example, the PDFF map is calculated through using a MR spectrum appropriate for use in the pelvic region, preferably with the MAGO or IDEAL algorithm as referenced above. T1, T2, T2* and ADC metrics are calculated by processing MR scan data with standard appropriate fitting algorithms. Corrected T1 would be standardized across scanner manufacturer and field strength and account for fat content in T1. These metrics can then be used for the generation of the corresponding MRI metric map.

The quantitative MRI metric maps will be used to determine tissue characteristics in the pelvic region. In an example this will include one or more of fat content and composition, iron content, inflammation, and alteration of the structure of tissue in the pelvic region.

In an example the quantitative MRI metric maps will also be used to aid in lesion identification and characterization of the endometrial tissue by highlighting areas with abnormal MRI metric values, which may correspond to areas with abnormal iron concentration.

By using one, or a combination of two or more of these MRI metric maps (such as T1, corrected T1, PDFF, T2, T2* ADC), metrics for endometrial tissue and other tissue in the pelvic region will be calculated.

In an example, pelvic tissue metrics may be based on a dictionary of both normal and abnormal tissue parametric values.

The methods as described above can be applied to the field of MRI, Quantitative MRI, pelvic MRI, gynaecological and fertility MRI investigation.

As described above, a method for analysing MRI data from the pelvic region to determine areas of endometriosis lesions is provided. In a further example there is also provided an apparatus for analysing MRI data from pelvic regions the apparatus comprising at least one processing component arranged to perform the above described methods. Preferably, the at least one processing component comprises one or more of: one or more programmable components arranged to execute computer program code for performing one or more of the steps of the method as previously described; and hardware circuitry arranged to perform one or more of the steps of the method as previously described. In a further preferred example the apparatus may further comprises at least one output component for outputting the pelvic map, or determined pelvic tissue heterogeneity characteristics the at least one output component comprising one or more of: a display device for displaying the pelvic map or determined pelvic tissue heterogeneity characteristics to a user; a data storage device for storing the pelvic map or determined pelvic tissue heterogeneity characteristics; and an interface component for transmitting the pelvic map or determined pelvic tissue heterogeneity characteristics to at least one external device.

As described above, an example of the method may be implemented in a computer program for running on an image processing system, at least including code portions for performing steps of a method according to an example when run on a programmable apparatus, such as an image processing system or enabling a programmable apparatus to perform functions of a device or system according to an example.

A computer program is a list of instructions such as a particular application program and/or an operating system. The computer program may for instance include one or more of: a subroutine, a function, a procedure, an object method, an object implementation, an executable application, an applet, a servlet, a source code, an object code, a shared library/dynamic load library and/or other sequence of instructions designed for execution on a computer system.

The computer program may be stored internally on a tangible and non-transitory computer readable storage medium or transmitted to the computer system via a computer readable transmission medium. All or some of the computer program may be provided on computer readable media permanently, removably or remotely coupled to an information processing system.

The tangible and non-transitory computer readable media may include, for example and without limitation, any number of the following: magnetic storage media including disk and tape storage media; optical storage media such as compact disk media (e.g., CD-ROM, CD-R, etc.) and digital video disk storage media; non-volatile memory storage media including semiconductor-based memory units such as FLASH memory, EEPROM, EPROM, ROM; ferromagnetic digital memories; MRAM; volatile storage media including registers, buffers or caches, main memory, RAM, etc.

A computer process typically includes an executing (running) program or portion of a program, current program values and state information, and the resources used by the operating system to manage the execution of the process. An operating system (OS) is the software that manages the sharing of the resources of a computer and provides programmers with an interface used to access those resources. An operating system processes system data and user input and responds by allocating and managing tasks and internal system resources as a service to users and programs of the system.

In the foregoing specification, examples have been described with reference to specific examples implementations. It will, however, be evident that various modifications and changes may be made therein without departing from the scope of the invention as set forth in the appended claims and that the claims are not limited to the specific examples described above.

Those skilled in the art will recognize that the boundaries between logic blocks are merely illustrative and that alternative embodiments may merge logic blocks or circuit elements or impose an alternate decomposition of functionality upon various logic blocks or circuit elements. Thus, it is to be understood that the architectures depicted herein are merely exemplary, and that in fact many other architectures can be implemented which achieve the same functionality.

Any arrangement of components to achieve the same functionality is effectively ‘associated’ such that the desired functionality is achieved. Hence, any two components herein combined to achieve a particular functionality can be seen as ‘associated with’ each other such that the desired functionality is achieved, irrespective of architectures or intermediary components. Likewise, any two components so associated can also be viewed as being ‘operably connected,’ or ‘operably coupled,’ to each other to achieve the desired functionality.

Furthermore, those skilled in the art will recognize that boundaries between the above described operations are merely illustrative. The multiple operations may be combined into a single operation, a single operation may be distributed in additional operations and operations may be executed at least partially overlapping in time. Moreover, alternative embodiments may include multiple instances of a particular operation, and the order of operations may be altered in various other embodiments.

However, other modifications, variations and alternatives are also possible. The specification and figures are, accordingly, to be regarded in an illustrative rather than in a restrictive sense.

In the claims, any reference signs placed between parentheses shall not be construed as limiting the claim. The word ‘comprising’ does not exclude the presence of other elements or steps than those listed in a claim. Furthermore, the terms ‘a’ or ‘an,’ as used herein, are defined as one or more than one. Also, the use of introductory phrases such as ‘at least one’ and ‘one or more’ in the claims should not be construed to imply that the introduction of another claim element by the indefinite articles ‘a’ or ‘an’ limits any particular claim containing such introduced claim element to inventions containing only one such element, even when the same claim includes the introductory phrases ‘one or more’ or ‘at least one’ and indefinite articles such as ‘a’ or ‘an.’ The same holds true for the use of definite articles. Unless stated otherwise, terms such as ‘first’ and ‘second’ are used to arbitrarily distinguish between the elements such terms describe. Thus, these terms are not necessarily intended to indicate temporal or other prioritization of such elements. The mere fact that certain measures are recited in mutually different claims does not indicate that a combination of these measures cannot be used to advantage.

1. A method of analyzing one or more MR images showing a pelvic region to identify features indicating areas that contain endometrial tissue comprising the steps of:
acquiring one or more quantitative non-contrast MR medical image scans of the pelvic region;
determining one or more MR metrics to determine one or more multiparametric MR map for the pelvic region; and
analyzing the multiparametric MR map to determine a biomarker indicating regions of abnormal iron concentration.

2. The method according to claim 1 wherein the one or more MR metrics comprise one or more of T1, corrected T1, T2, T2*, PDFF and ADC.

3. The method as claimed in claim 1 wherein the biomarker is determined as one or more of: a T2* value greater than 15 ms; a T1 value less than 1000 ms.

4. The method as claimed in claim 2 wherein the one or more MR metrics further comprises a deoxyhaemoglobin/oxyhaemoglobin ratio, BOLD/R2*.

5. The method as claimed in any preceding claim 1 wherein the determined metrics generate an MRI map for each of the determined metrics.

6. The method as claimed in claim 1 wherein at least two of the determined metrics are combined to generate a further metric map, and this further metric map is used in a calculation of a pelvic composite map.

7. The method as claimed in claim 6 wherein raw values for the at least two determined metrics are used to generate the further metric map.

8. The method as claimed in claim 1 wherein the regions of abnormal iron concentration indicate a presence of endometrial tissue in the pelvic region.

9. The method as claimed in claim 3 further comprising determining an age of the endometrial tissue using the T2* value.

10. The method as claimed in claim 1 wherein the one or more MR medical scan images are obtained at a clinical magnetic field strength.

11. The method as claimed in claim 9 wherein the field strength is at least 1.5T.

12. The method as claimed in claim 10 wherein the field strength is 3T.

13. The method as claimed in claim 1 wherein the MRI scan(s) is/are 2D scan(s) which produces a set of 2D images comprised of pixels, or 3D scan(s) with a 3D image comprised of voxels.

14. The method as claimed in claim 1 wherein the MRI metric map is a PDFF map or a corrected T1 map.

15. The method as claimed in claim 1 wherein at least one MRI metric is combined with one or more other MRI metrics, before generation of an additional MRI metric map which provides an improved estimate of an initial value of at least one MRI metric.

16. The method as claimed in claim 15 wherein the at least one metric is a T1 metric and is combined with at least one of PDFF and T2*.

17. The method as claimed in claim 1 further comprising the step of generating at least one of the following MRI metric maps: T1 map, T2 map, T2* map, ADC map.

18. The method as claimed in claim 1 wherein the T1 metric is determined using a Modified Look Locker Inversion recovery sequence acquisition, shortened Modified Look Locker Inversion recovery sequence acquisition or a variable flip angle sequence acquisition.

19. The method as claimed in claim 1 wherein the T2* metric and/or the PDFF metric are determined using a multi echo gradient echo sequence acquisition.

20. The method as claimed in claim 1 wherein the T2 metric is determined using a multi-contrast spin echo sequence acquisition.

21-27. (canceled)
